(19) United States
(12) Patent Application Publication
(10) Pub. No.: US 2010/0048452 A1
(43) Pub. Date: Feb. 25, 2010

(54) Injectable Sustained-Release Formulation Of Active Principles, And Process For The Preparation Thereof

(75) Inventors: Jerome Gaucheron, Castres (FR); Gerard Trouve, Castres (FR); Jerome Aucouturier, Chatenay-Malabry (FR)

Correspondence Address: AIR LIQUIDE;Intellectual Property, 2700 POST OAK BOULEVARD, SUITE 1800, HOUSTON, TX, 77056, US

(73) Assignee: Societe D'Exploitation De Produits Pour Les Industries Chimiques, paris (FR)

(21) Appl. No.: 12/307,849

(22) PCT Filed: Jul. 3, 2007
(86) PCT No.: PCT/FR2007/051584

§ 371 Date: Sep. 1, 2009

(30) Foreign Application Priority Data
Jul. 12, 2006 (FR) 0652929

(51) Int. Cl. A61K 9/107; A61K 38/02

(52) U.S. Cl. 514/2; 514/784; 514/786

(57) ABSTRACT

Composition for the implementation of a therapeutic method for the human or animal body, comprising a fatty phase (O) and an aqueous phase (W), in the form of an 5 emulsion of the water-in-oil (W/O) type, in which said aqueous phase (W) comprises at least one hydrosoluble active ingredient, characterized in that said composition has a viscosity, measured at 25° C. in a 250-cm³ beaker having a diameter of about 7 cm, using a Brookfield LVT viscosimeter equipped with a No. 2 spindle turning at a speed of 30 or 60 revolutions pre minute, of less than or equal to 10 [sic] 2000 mPas, and preferably less than 200 mPas.

The invention relates to a novel formulation of pharmaceutical or veterinary active principles, for promoting their sustained release over time into the human or animal body.

The sustained release of medicaments is a method for sustaining over time the therapeutic effects of an active principle, for reducing its side effects and for minimizing the risks associated with temporary exceeding of the toxicity threshold in the patient. Many formulations for the oral, topical or injectable sustained release of medicaments have been described in the literature. Among these formulations, solid injectable forms and liquid injectable forms are distinguished.

Solid injectable forms are suspensions of microbeads or nanobeads with a mean diameter of between 0.01 μm and 100 μm, in an isotonic aqueous solution. Such suspensions often have a tendency to sediment and are therefore relatively unstable on storage, and the homogeneity of the medicament is not always ensured, which may pose problems as regards ensuring the administration of a precise dose of active principle to the patient. The microbeads or nanobeads of these suspensions are generally constituted of biodegradable polymers such as lactic acid and/or glycolic acid copolymers, as described in the US patent application published under the number 2005/048 115 A1. Major drawbacks in the use of these microbeads or nanobeads include the use for preparing them of volatile organic solvents that are hazardous to human health, and also the residual presence of toxic monomers in the final formulation. Other authors have described microbeads of fatty acids that are solid at room temperature as injectable active-principle vectors, such as those described in the international patent application published under the number WO 94/26252. In this case, the drawbacks lie in the fact that the suspensions can only be sparingly charged with microbeads, and thus with active principle, in order to remain fluid and injectable. Furthermore, changes in the crystalline form of the fatty substances over time, or during cold storage, have been observed, which leads to unsatisfactory release profiles of the active principles also described. Finally, such microbeads can only be used with lipophilic active principles.

Stable, homogeneous and fluid liquid injectable forms are preferred for injectable administration. One particularly useful form is an emulsion, which is a mixture of an aqueous phase and a liquid lipid phase, stabilized with surfactants. When the lipid phase is dispersed in the aqueous phase, an emulsion of “oil-in-water” type, written O/W, is obtained. Such emulsions have been used for a long time for parenteral feeding and have also been proposed for the injection of lipophilic active principles to ensure sustained release thereof in the human body. However, such emulsions use fatty phases such as plant oils or medium-chain triglycerides, which have low solvent power and only make it possible to dissolve small amounts of carefully selected medicaments.

Since the majority of active principles are water-soluble, it has been attempted to use emulsions of “water-in-oil” type, written W/O, for which the aqueous phase is dispersed in a lipid phase that acts as a barrier or membrane, isolating the medicament from the biological fluids of the human body and allowing its gradual release over time. However, emulsions of “water-in-oil” type are often too viscous and are thus difficult to inject by syringe; when they are injected, they cause local reactions at the site of injection, due both to the viscosity and the oily nature of the continuous phase; finally, they are sparingly stable over time.

The international patent application published under the number WO 02/067 899 discloses compositions in the form of water-in-oil (W/O) emulsions for vaccines, preferably having a viscosity of less than 100 mPa·s, in which the emulsifier is, for example, Arcacel™ P135 [polyester emulsifier of poly(12-hydroxystearic acid) type] in which the aqueous phase comprises at least one water-soluble active principle.

The international patent application published under the number WO 96/40057 discloses a medicament in the form of an inverse emulsion of water-in-oil type, of low viscosity, the fatty phase comprising a fluorocarbon-based oil, and the aqueous phase at least one active principle.

This is why the inventors set themselves the aim of developing a water-in-oil emulsion of active principles that does not have the drawbacks outlined above.

According to a first aspect, a subject of the invention is a composition for performing a therapeutic method on the human or animal body, comprising a fatty phase (O) and an aqueous phase (W), in the form of an emulsion of water-in-oil (W/O) type that may be injected into said human or animal body, in which said aqueous phase (W) comprises at least one pharmaceutical or veterinary water-soluble active principle, characterized in that said fatty phase (O) comprises one or more surfactants with an overall HLB value of between 3 and 8, chosen from fatty acid esters of sorbitol or mannitol or fatty acid esters of sorbitan or mannitan, (poly)alkoxylated fatty acid triglycerides, (poly)alkoxylated polyglycerol esters of fatty acids and said composition has a viscosity measured at 25° C. in a 250 cm³ beaker with a diameter of about 7 cm, using a Brookfield LVT viscometer with a No. 2 spindle rotating at a speed of 30 or 60 rpm, of less than or equal to 200 mPa·s.

For the purposes of the present invention, the HLB value of a surfactant of the fatty acid ester family is calculated by the formula HLB=20 (1−Iₛ/Iₐ), in which Iₛ represents the saponification number and Iₐ the acid number of said surfactant. These two numbers, the saponification number and the acid number, are determined via methods described in the European Pharmacopea. The HLB value of surfactants forming part of another chemical family is calculated by the general formula HLB=5E/M in which E represents the molar mass of the hydrophilic portion of the surfactant and M the total molar mass of said surfactant. In the case of a mixture of surfactants, the overall HLB value of the mixture is the weight sum of the HLB values of each surfactant.

The constituent fatty phase of the composition that is the subject of the present invention, which must be liquid at 4° C., generally comprises one or more compounds chosen from oils of mineral, plant or animal origin, alkyl esters of said oils, alkyl esters of fatty acids or alkyl ethers of fatty alcohols, esters of fatty acids and of polyols and ethers of fatty alcohols and of polyols.

Examples of oils of mineral origin include oils of petroleum origin, for instance white mineral oils, such as Marcol™ 52, Marcol™ 82 or Drakeol™ 6 VR. Examples of oils of plant origin include groundnut oil, olive oil, sesame seed oil, soybean oil, wheatgerm oil, grapeseed oil, sunflower oil, castor oil, linseed oil, soybean oil, corn oil, coconut oil, palm oil, walnut oil, hazelnut oil and rapeseed oil, or alternatively olive squalane or squalene. Examples of oils of animal origin include spermaceti oil, tallow oil, squalane or squalene extracted from shark liver, and fish oils.

Examples of alkyl esters of oils include the methyl, ethyl, linear or branched propyl and linear or branched butyl esters of said oils.

Fatty acids that are suitable for preparing the esters mentioned above more particularly include those containing from 12 to 22 carbon atoms, for instance myristic acid, palmitic acid, oleic acid, ricinoleic acid or isostearic acid, and advantageously a fatty acid that is liquid at 20° C.

Examples of fatty acid esters or of fatty alcohol ethers include alkyl esters of fatty acids, such as ethyl oleate, methyl oleate, isopropyl myristate or octyl palmitate, esters of fatty acids and of polyols or ethers of fatty alcohols and of polyols, such as fatty acid monoglycerides, fatty acid diglycerides, fatty acid triglycerides, esters of fatty acids with a polyglycerol or fatty acid esters of propylene glycol, and more particularly esters of fatty acids with a hexol, for instance sorbitol or mannitol, and esters of fatty acids with a hexol anhydride, for instance sorbitan or mannitan.

In the context of the present invention, the fatty phase may comprise only one of the compounds mentioned above, or a mixture of several of the compounds mentioned above.

According to a first particular aspect of the present invention, the constituent fatty phase (O) of the pharmaceutical composition is chosen from white mineral oils, fluid liquid paraffins, squalane, squalene and ethyl oleate, or a mixture of these oils.

The fatty phase comprises, per 100% of its mass, between about 1% and 15% by mass and preferably between 3% and 10% by mass of surfactants.

A subject of the invention is more particularly a composition as defined previously in which the surfactant or the mixture of surfactants has an overall HLB value of greater than or equal to 5 and less than 8.

The surfactants used are generally chosen from modified fatty substances. The modified fatty substances used in the context of the present invention may be of mineral, plant or animal origin. Modified fatty substances of mineral origin include oils of petroleum origin. Modified fatty substances of plant origin include modified plant oils, for example modified groundnut oil, olive oil, sesame seed oil, soybean oil, wheatgerm oil, grapeseed oil, sunflower oil, castor oils linseed oil, soybean oil, corn oil, coconut oil, palm oil, walnut oil, hazelnut oil or rapeseed oil. Modified fatty substances of animal origin include, for example, modified squalane, modified squalene, modified spermaceti oil and modified tallow oil. The term “modified fatty substances” especially denotes the carboxyl, sulfate, phosphate or alkoxy derivatives of fatty substances and more particularly the (poly)alkoxylated derivatives of oils or the (poly)alkoxylated derivatives of alkyl esters of oils and more particularly the (poly)ethoxylated and/or (poly)propoxylated derivatives of oils or the (poly)ethoxylated and/or (poly)propoxylated derivatives of methyl, ethyl, linear or branched propyl or linear or branched butyl esters of said oils.

A subject of the invention is, more specifically, a composition as defined previously, in which the modified fatty substance is chosen from ethoxylated derivatives of oils with a mean degree of ethoxylation of between 1 and 10 (also referred to as an EO number of between 1 and 10). The term “modified fatty substances” also denotes the esters of fatty acids and of polyols or the ethers of fatty alcohols and of polyols, and more particularly the esters of fatty acids with a hexol, for instance sorbitol or mannitol, or the esters of fatty acids with a hexol anhydride, for instance sorbitan or mannitan, the (poly)alkoxylated derivatives of esters of fatty acids and of polyols or the (poly)alkoxylated derivatives of ethers of fatty alcohols and of polyols, for instance (poly)alkoxylated fatty acid triglycerides, (poly)alkoxylated esters of fatty acids and of polyglycerol, and more particularly the (poly)alkoxylated esters of fatty acids with a hexol, for instance sorbitol or mannitol, or the (poly)alkoxylated esters of fatty acids with a hexol anhydride, for instance sorbitan or mannitan, having a mean degree of ethoxylation of between 1 and 40 (also referred to as an EO number of between 1 and 40) and preferably between 5 and 20.

In the context of the present invention, the term “esters of fatty acids and of polyols” denotes fatty acid monoesters of polyols or fatty acid polyesters of polyols, for instance fatty acid diesters of polyols or fatty acid triesters of polyols. The same is likewise true for the (poly)alkoxylated derivatives of said esters.

In the context of the present invention, the term “ethers of fatty alcohols and of polyols” denotes the monoethers of fatty alcohols and of polyols or the polyethers of fatty alcohols and of polyols, for instance the diethers of fatty alcohols and of polyols or the triethers of fatty acids and of polyols. The same is likewise true for the (poly)alkoxylated derivatives of said ethers.

A subject of the invention is more particularly a composition as defined previously, in which the modified fatty substances are chosen from (poly)ethoxylated derivatives of esters of fatty acids and of polyols or (poly)ethoxylated derivatives of ethers of fatty alcohols and of polyols, and more particularly (poly)ethoxylated esters of fatty acids with glycerol or with a hexol, for instance sorbitol or mannitol, or (poly)ethoxylated esters of fatty acids with a hexol anhydride, for instance sorbitan or mannitan with a mean degree of ethoxylation of between 5 and 10 (also referred to as an EO number of between 5 and 10).

Fatty acids that are suitable for preparing the modified fatty substances described above include those containing on average from 12 to 22 carbon atoms, for instance those containing from 16 to 18 carbon atoms, for instance oleic acid, ricinoleic acid, hydroxystearic acid or isostearic acid, and advantageously fatty acids that are liquid at 20° C. The composition as defined above more particularly contains one or more modified fatty substances as described above, derived from oleic acid.

According to another particular aspect of the present invention, the surfactant or the mixture of surfactants present in the fatty phase consists essentially of one or more esters chosen from mannitan esters, sorbitan esters, (poly)alkoxylated mannitan esters and (poly)alkoxylated sorbitan esters. Most particularly, the surfactant present in the fatty phase consists of a mixture of mannitan oleate and of (poly)ethoxylated mannitan oleate, a mixture of sorbitan oleate and of (poly)ethoxylated sorbitan oleate, a mixture of sorbitan oleate and of (poly)ethoxylated mannitan oleate or a mixture of mannitan oleate and of (poly)ethoxylated sorbitan oleate, said mixtures having an overall HLB value of greater than or equal to 3 and less than 8.

According to another particular aspect of the present invention, the surfactant or the mixture of surfactants present in the fatty phase consists essentially of one or more compounds chosen from lecithins, for instance soybean lecithin or egg lecithin, hydrogenated lecithins, phospholipids and sphingolipids.

The composition as defined above comprises, per 100% of its mass, generally up to 50% by mass of aqueous phase, the aqueous phase consisting of water or of any bioavailable aqueous solvent, such as water buffered with phosphate buffer supplemented with the hydrophilic active principle(s) to be injected.

Preferably, the composition as defined previously is used in a curative therapeutic treatment method. Hydrophilic active principles that are suitable for such an injectable formulation include, for example, water-soluble anti-cancer active principles, hormones, antibiotics, antiviral agents, analgesic active agents, vasodilators, antidiabetic active agents, anesthetics, sedatives, contrast agents, immunomodulators, anti-hemophilic factors, neuroleptic agents, nutritional active agents, steroids, thrombolytic agents and biopharmaceutical active agents, for instance recombinant proteins.

According to another aspect, a subject of the invention is a process for preparing a composition as defined previously, characterized in that it comprises the following successive steps:
a step (a) of dissolving the hydrophilic principle in water or the pharmacologically acceptable aqueous solvent;
a step (b) of mixing the aqueous phase prepared in step (a) with a fatty phase containing a surfactant or a mixture of surfactants.

According to a final aspect, a subject of the invention is a process for the sustained release of an active principle in the human or animal body, characterized in that said active principle is administered by injection in the form of a water-in-oil emulsion as defined previously.

The examples that follow illustrate the invention without, however, limiting it.

1—Preparation of Caffeine W/O Emulsions

Caffeine is predissolved at the desired concentration, in particular of 1 mg/mL, but more generally from 0.1 to 10 mg/mL, in physiological water, the necessary amounts of oil and of necessary surfactants are stirred until a homogeneous mixture is obtained. In all the examples described, the proportions of fatty phase (oil plus surfactants) and of aqueous caffeine solution are maintained equal to 70% of fatty phase and 30% by mass of aqueous phase. To prepare the emulsion, the aqueous phase is poured into the oily phase and the mixture is stirred for 3 minutes using a laboratory rotor-stator stirrer of Silverson™ L4R type. Six samples of W/O emulsions were prepared with caffeine as hydrophilic active principle and the ingredients indicated in the following tables:

The mixtures of surfactants used are characterized by their physicochemical specifications, as indicated in the table below.

| | Surfactant | | |
|---|---|---|---|
| | No. 1 | No. 2 | No. 3 |
| Nature | Mannitan | Modified | Mannitan oleate and |
| | oleate | mannitan | (poly)ethoxylated |
| | | oleate | plant oil |
| Saponification | 164-172 | 147-160 | 157-170 |
| number | | | |
| Hydroxyl | 89-100 | 90-105 | 87-100 |
| number | | | |
| Refractive | 1.475-1.477 | 1.475-1.476 | 1.474-1.479 |
| index | | | |
| HLB value | 3 | 5 | 3.5 |
| (calculated) | | | |

The caffeine emulsions obtained have the following characteristics:

| | Viscosity | | | |
|---|---|---|---|---|
| | (Brookfield | | | |
| | LVT, spindle | | Surfactant | Aqueous |
| W/O | No. 2, speed | Oils of the | nature/mass | phase |
| emulsion | 30 rpm) | fatty phase | percentage | (mass %) |
| Emulsion | 70 | Squalane- | | 30% |
| No. 1 | | squalene | | |
| | | mixture (50%-50% | | |
| | | by mass) | | |
| Emulsion | 60 | Squalane- | | 30% |
| No. 2 | | squalene | | |
| | | mixture (5%-95% | | |
| | | by mass) | | |
| Emulsion | 400 | Squalane | No. 1/20% | 30% |
| No. 3 | | | | |
| Emulsion | 750 | Liquid paraffin | No. 1/11.5% | 30% |
| No. 4 | | | | |
| Emulsion | 100 | Liquid paraffin | No. 2/11.5% | 30% |
| No. 5 | | | | |
| Emulsion | 40 | Ethyl | No. 3/11.5% | 30% |
| No. 6 | | oleate/squalane | | |

2—In Vitro Evaluation of the Emulsions as Regards Their Capacity for Sustained Release of Active Principles

The sample of the emulsion to be evaluated is deposited on a “receiving” aqueous phase, contained in the container of the Erweka™ DT 600 dissolution machine; the whole is mixed and a sample of this phase is taken regularly (t=0, 24 hours, 48 hours, 72 hours, 96 hours) in order to assay the active agent released therefrom into the receiving phase. The results obtained are given in the following table in terms of percentage of caffeine released:

| | | t = | t = | t = | t = |
|---|---|---|---|---|---|
| | t = 0 | 24 hours | 48 hours | 72 hours | 96 hours |
| Emulsion | 0% | 30% | 35% | 40% | 45% |
| No. 1 | | | | | |
| Emulsion | 0% | 60% | 80% | 90% | 100% |
| No. 2 | | | | | |
| Emulsion | 0% | 30% | 40% | 50% | 60% |
| No. 3 | | | | | |
| Emulsion | 0% | 25% | 35% | 40% | 45% |
| No. 4 | | | | | |
| Emulsion | 0% | 40% | 60% | 75% | 85% |
| No. 5 | | | | | |
| Emulsion | 0% | 90% | 100% | 100% | 100% |
| No. 6 | | | | | |

3—In Vivo Evaluation of the Emulsions According to the Invention as Regards Their Capacity for Sustained Release of Active Principles

The pharmacokinetics of caffeine were evaluated on four groups of 4 animals by subcutaneous injection of emulsions No. 1 and 2, of a physiological caffeine solution (SC solution), and of a physiological caffeine solution intravenously (IV solution). For these four groups, the dose of caffeine injected is 40 mg/kg of rat.

The caffeine concentration in the blood (in μg/ml of blood) is determined periodically, and the following results are obtained:

| | t in minutes | | | | | | | |
|---|---|---|---|---|---|---|---|---|
| | t = 0 | t = 60 | t = 120 | t = 180 | t = 240 | t = 360 | t = 480 | t = 600 |
| Emulsion No. 1 | 0 | 25 | 32 | 30 | 27 | 20 | 15 | 100 |
| Emulsion No. 2 | 0 | 35 | 35 | 30 | 27 | 20 | 15 | 10 |
| SC solution | 0 | 45 | 45 | 35 | 32 | 25 | 15 | 10 |
| IV solution | 0 | 45 | 35 | 35 | 32 | 25 | 15 | 10 |

These results give the following pharmacokinetic data:

| | Emulsion | Emulsion | SC | IV |
|---|---|---|---|---|
| | No. 1 | No. 1 | solution | solution |
| AUC from 0 to | 208 | 247 | 279 | 241 |
| 10 hours | | | | |
| (h · μg/cm³) | | | | |
| Rate of | 1.38 | 1.31 | 4.46 | / |
| absorption (h) | | | | |
| Time to reach | 1.76 | 1.75 | 0.78 | / |
| Cₘₐₓ (h) | | | | |
| Estimated Cₘₐₓ | 31.6 | 39.1 | 48.7 | / |
| (μg/ml) | | | | |
| Half-life of | 4.29 | 3.8 | 4.44 | 4.22 |
| elimination (h) | | | | |

AUC: area under the curve, i.e. the measurement of the area under the curve plotted by placing the time (in hours, h) on the x-axis, and the concentration in μg/mL of blood on the y-axis.

The results show that the caffeine passes more slowly into the blood when it is injected in the form of a subcutaneous emulsion, when compared with an aqueous solution injected in the same manner, while conserving areas under the curves equivalent to that corresponding to an intravenous injection.

The absorption phase of the caffeine in emulsion form is three times slower and the time to reach the maximum plasmatic concentration is delayed by one hour. The maximum plasmatic concentrations obtained with the emulsions according to the invention are 20% to 35% lower than those obtained with the caffeine solution.

1-9. (canceled)

10. A composition for performing a therapeutic method on a human or animal body, the composition comprising a fatty phase (O) and an aqueous phase (W), in the form of an emulsion of water-in-oil (W/O) type that may be injected into said human or animal body, said aqueous phase (W) comprising at least one pharmaceutical or veterinary water-soluble active principle and said fatty phase (O) comprising one or more surfactants with an overall HLB value of between 3 and 8, chosen from fatty acid esters of sorbitol or mannitol or fatty acid esters of sorbitan or mannitan, (poly)alkoxylated fatty acid triglycerides, (poly)alkoxylated polyglycerol esters of fatty acids and said composition having a viscosity of less than or equal to 200 mPa·s.

11. The composition of claim 10, wherein said fatty phase (O) is chosen from white mineral oils, fluid liquid paraffins, squalane, squalene and ethyl oleate, or a mixture of these oils.

12. The composition of claim 10, wherein said fatty phase (O) comprises, per 100% of its mass, between 1% and 15% by mass of surfactants.

13. The composition of claim 12, wherein said fatty phase (O) comprises, per 100% of its mass, between 3% and 10% by mass of surfactants.

14. The composition of claim 12, wherein the surfactants are chosen from esters of fatty acids and of polyols.

15. The composition of claim 14, wherein the surfactants are chosen from (poly)alkoxylated esters of fatty acids and of glycerol, sorbitol or mannitol, or (poly)alkoxylated esters of fatty acids and of sorbitan or mannitan, with a mean degree of ethoxylation of between 1 and 40 and preferably between 5 and 10.

16. The composition of claim 15, wherein the surfactants are chosen from mixtures of mannitan oleate and of (poly)ethoxylated mannitan oleate, mixtures of sorbitan oleate and of (poly)ethoxylated sorbitan oleate, mixtures of sorbitan oleate and of (poly)ethoxylated mannitan oleate or mixtures of mannitan oleate and of (poly)ethoxylated sorbitan oleate, said mixtures having an overall HLB value of greater than or equal to 3 and less than 8.

17. The composition of claim 12, wherein the surfactants are chosen from lecithins, such as soybean lecithin or egg lecithin, hydrogenated lecithins, phospholipids and sphingolipids.

18. The composition of claim 10, wherein the composition comprises, per 100% of its mass, at least 30% by mass of aqueous phase.

19. The composition of claim 10, wherein the water-soluble active principle is chosen from anti-cancer active principles, hormones, immunomodulators, anti-hemophilic factors, neuroleptic agents, nutritional active agents, steroids, thrombolytic agents and pharmaceutical active agents.

20. The composition of claim 19, wherein the pharmaceutical active agents are recombinant proteins.

21. The composition of claim 10, wherein said fatty phase (O) comprises, per 100% of its mass, between 3% and 10% by mass of surfactants and the surfactants are chosen from (poly)alkoxylated esters of fatty acids and of glycerol, sorbitol or mannitol, or (poly)alkoxylated esters of fatty acids and of sorbitan or mannitan, with a mean degree of ethoxylation of between 1 and 40 and preferably between 5 and 10.

22. The composition of claim 21, wherein the viscosity is measured at 25° C. in a 250 cm³ beaker with a diameter of about 7 cm, using a Brookfield LVT viscometer with a No. 2 spindle rotating at a speed of 30 or 60 rpm.

23. The composition of claim 22, wherein the composition comprises, per 100% of its mass, at least 30% by mass of aqueous phase.

24. The composition of claim 23, wherein the water-soluble active principle is chosen from anti-cancer active principles, hormones, immunomodulators, anti-hemophilic factors, neuroleptic agents, nutritional active agents, steroids, and thrombolytic agents and pharmaceutical active agents.

25. The composition of claim 24, wherein the pharmaceutical active agents are recombinant proteins.

26. The composition of claim 10, wherein said fatty phase (O) comprises, per 100% of its mass, between 3% and 10% by mass of surfactants and the surfactants are chosen from lecithins such as soybean lecithin or egg lecithin, hydrogenated lecithins, phospholipids and sphingolipids.

27. The composition of claim 26, wherein the viscosity is measured at 25° C. in a 250 cm³ beaker with a diameter of about 7 cm, using a Brookfield LVT viscometer with a No. 2 spindle rotating at a speed of 30 or 60 rpm.

28. The composition of claim 27, wherein the composition comprises, per 100% of its mass, at least 30% by mass of aqueous phase.

29. The composition of claim 28, wherein the water-soluble active principle is chosen from anti-cancer active principles, hormones, immunomodulators, anti-hemophilic factors, neuroleptic agents, nutritional active agents, steroids, and thrombolytic agents and pharmaceutical active agents.

30. The composition of claim 29, wherein the pharmaceutical active agents are recombinant proteins.

31. The composition of claim 10, wherein the viscosity is measured at 25° C. in a 250 cm³ beaker with a diameter of about 7 cm, using a Brookfield LVT viscometer with a No. 2 spindle rotating at a speed of 30 or 60 rpm.

32. A process for preparing a composition for performing a therapeutic method on a human or animal body, comprising a fatty phase (O) and an aqueous phase (W), in the form of an emulsion of water-in-oil (W/O) type that may be injected into said human or animal body, said aqueous phase (W) comprising at least one pharmaceutical or veterinary water-soluble active principle and said fatty phase (O) comprising one or more surfactants, with an overall HLB value of between 3 and 8, chosen from fatty acid esters of sorbitol or mannitol or fatty acid esters of sorbitan or mannitan, (poly)alkoxylated fatty acid triglycerides, (poly)alkoxylated polyglycerol esters of fatty acids and said composition has a viscosity of less than or equal to 200 mPa·s., wherein the process comprises the following steps:
(a) dissolving the hydrophilic principle in water or a pharmacologically acceptable aqueous solvent; and
(b) mixing the aqueous phase prepared in step (a) with a fatty phase containing a surfactant or a mixture of surfactants.
